(19) United States
(12) Patent Application Publication
(10) Pub. No.: US 2011/0004104 A1
(43) Pub. Date: Jan. 6, 2011

(54) DEVICE AND METHOD FOR MICRO-ELASTOGRAPHY

(76) Inventor: Laurent Sandrin, I'Hay-les-Roses (FR)

Correspondence Address: PILLSBURY WINTHROP SHAW PITTMAN, LLP, P.O. BOX 10500, MCLEAN, VA, 22102, US

(21) Appl. No.: 12/919,747

(22) PCT Filed: Feb. 27, 2009
(86) PCT No.: PCT/FR2009/000218

§ 371 Date: Aug. 26, 2010

(30) Foreign Application Priority Data
Feb. 29, 2008 (FR) 0851345

(51) Int. Cl. A61B 8/14

(52) U.S. Cl. 600/459

(57) ABSTRACT

Device and method associated with vibration micro-elastography for qualitative and/or quantitative measurements of viscoelastic properties, such as the elasticity and/or the viscosity of a viscoelastic medium and more particularly of a human or animal biological tissue, carried out inside the human or animal body. This method may be carried out with the aid of a probe inserted into the human or animal body, which probe is connected to an external controller via a threadlike tube.

The present invention relates to a device and a method for measurement of viscoelastic properties, referred to as VPs hereinafter, of a viscoelastic medium and, more particularly, of a human or animal biological tissue.

The present invention relates, more particularly, to a method and a device for micro-elastography, referred to as a MED hereinafter, for quantitative and qualitative measurements of viscoelastic properties, such as the elasticity and/or the viscosity of a human or animal biological tissue implemented inside the human or animal body.

In order to measure the VPs of a biological tissue it is known to use impulse elastometry, as disclosed for example in patent application FR 2843290 filed on 8 Aug. 2002 in the name of ECHOSENS, a public limited company.

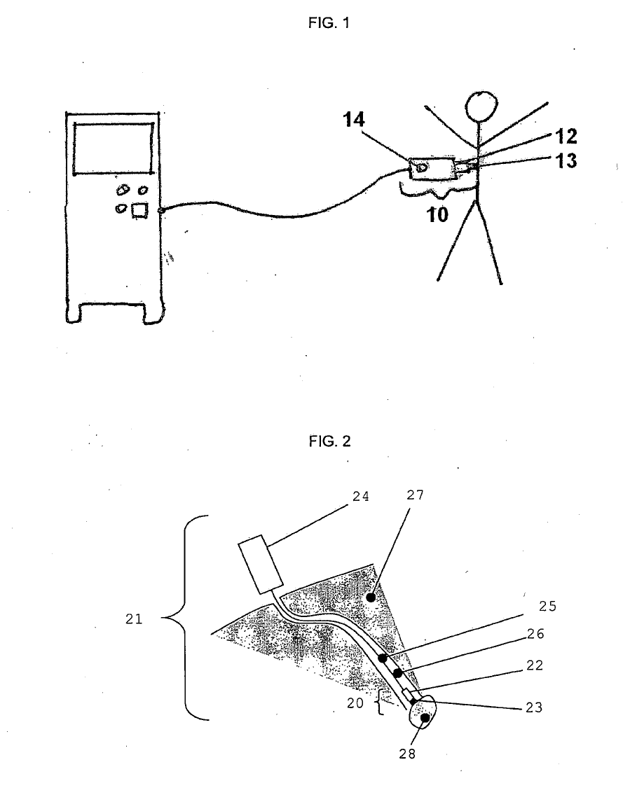

A method of this type is implemented using a probe 10 (FIG. 1) equipped with a vibration generator 12 generating a low-frequency elastic wave in a tissue, for example by vibration of a sensor, and analysing the propagation of this low-frequency wave with the aid of ultrasonic waves emitted and received by an ultrasonic transducer 13 during propagation of the low-frequency elastic wave. It should be noted that, in this implementation, the ultrasonic transducer vibrates upon contact with tissues.

The probe 10 equipped with the vibration generator 12 and with the ultrasonic transducer 13 also has a controller 14 controlling said vibration generator and ultrasonic transducer.

This method makes it possible to measure the VPs of an organ arranged in the vicinity of the epidermis, against which the probe 10 is placed.

A method and a device of this type do pose drawbacks. In particular, they do not permit measurement of tissues belonging to organs arranged deep within the human body. In fact, the propagation of the low-frequency elastic wave(s) in a body is all the more disturbed by the heterogeneity of said body as this/these wave(s) advance deeply into the body.

Furthermore, when the low-frequency wave is generated, diffraction close to its source (the vibration generator) is produced over a depth that depends, inter alia, on the size of this source, the VPs of the medium and the frequency of the low-frequency elastic wave.

This minimum depth, below which it is not possible to take a measurement, is approximately 10 mm for a centre frequency of the low-frequency elastic wave of 50 Hz. Data abstraction is carried out over this depth when calculating the VPs in order to minimise this problem.

Furthermore, a method of this type is confronted with the problem of adipose tissues sandwiched between the epidermis and the tissue to be measured when it is applied to a body. In fact, adipose tissues deform and weaken high-frequency ultrasonic waves and low-frequency elastic waves, which makes it difficult to observe these waves beyond a maximum observation depth. An adipose layer more than 25 mm thick thus prevents measurement of VPs of tissues arranged beneath this adipose layer.

An additional drawback is the need for the practitioner to manually maintain satisfactory contact between the epidermis and the device, and in particular its ultrasonic transducer. Indeed, in order to obtain optimal propagation the end of the ultrasonic transducer must be perpendicular to the tissue of which the VPs are to be measured, and any variations in contact may spoil implementation of the method.

In order to overcome at least one of the above-mentioned drawbacks, the present invention relates to a device for vibration elastography for quantitative and/or qualitative measurement of viscoelastic properties of a human or animal tissue, this device being, equipped with:
a probe, comprising at least one ultrasonic transducer and a low-frequency vibration generator, the ultrasonic transducer(s) generating ultrasonic waves making it possible to analyse the propagation of low-frequency elastic waves propagating in the organ and generated by the low-frequency vibration generator, the probe being intended to be positioned near to or against the organ,
a controller, connected to the probe, comprising means for actuating the probe, the controller being intended to be kept outside the human or animal body,
means for mechanically connecting the probe to the controller, the mechanical connection means being formed by a threadlike tube.

In particular, a threadlike tube is understood to mean an elongate and thin sheath, pipe or cannula, that is to say having a small diameter or of small thickness, this threadlike tube possibly being supple, flexible or rigid.

A device of this type, referred to as a MED for micro-elastography device, makes it possible to quantitatively and/or qualitatively measure the VPs of a deep human or animal tissue, that is to say inside the human or animal body, by leading the probe equipped with at least one ultrasonic transducer and a vibration generator near to or against this deep tissue, thus making it possible to overcome the heterogeneity of the human or animal body and also the thickness of the adipose layer.

Said threadlike tube is preferably longer than 20 mm, and is preferably between 20 mm and 3 metres long.

The threadlike tube connecting the internal probe to the external vibration generator is advantageously flexible and free from any angular or rigid stress.

Furthermore, the threadlike tube is the vibration generator in accordance with one advantageous possibility offered by the invention.

The ultrasonic transducer advantageously has an active diameter, corresponding to the diameter of ultrasonic emission and acquisition, of less than 3 mm.

According to a preferred embodiment of the invention, said device comprises a threadlike tube formed by a catheter, a needle or an endoscope, of which the distal end contains the probe and the proximal end comprises the controller.

In addition, depending on the field of investigation, endoscopes may be flexible or rigid and are therefore referred to as: bronchoscopes, gastroscopes, duodenoscopes, rectoscopes, laparoscopes, arthroscopes, etc.

Likewise, needles may be flexible or rigid and may be biopsy needles, radiofrequency needles, etc.

According to a preferred embodiment of the invention said threadlike tube forms a hollow shaft, into which a needle is inserted.

Said controller advantageously comprises means for controlling the transfer of energy to the vibration generator and/or to the ultrasonic transducer(s).

The present invention also relates to a method for vibration elastography for the quantitative and/or qualitative measurement of viscoelastic properties of a human or animal tissue, implementing a device according to any one of claims 1 to 12 and consisting of:
positioning the probe near to or against the tissue to be measured,
keeping in contact with the tissue at least one ultrasonic transducer whilst the step of ultrasonic emission and acquisition and of generation of one or more low-frequency elastic wave(s) is carried out,
generating one or the low-frequency elastic wave(s),
generating, at the same time as the preceding step, ultrasonic emissions and acquiring, at high-speed, high-frequency ultrasonic signals during propagation of the low-frequency elastic wave or waves,
calculating the spatio-temporal variations of displacements and/or deformations and/or speeds of displacement and/or speeds of deformation produced in the organ or, more generally, of any movement parameter,
calculating the viscoelastic properties of the tissue.

To this end the probe and a portion of the threadlike tube are inserted via natural routes (airways, the mouth, nose, rectum, etc.), by percutaneous route, that is to say through the skin, via artificial routes (cannula, surgical retractor, trocar, etc.), or via material routes (operating channel of an endoscope, catheter, etc.) so as to be transported near to or directly against the tissue of which the VPs are established.

Said threadlike tube inserted into the operating channel of an endoscope is advantageously oriented by an erector equipping an endoscope so as to position the distal end of the device in front of the tissues to be measured.

Said probe and a portion of the threadlike tube are advantageously inserted into a liquid belonging to the human or animal body.

The high-frequency ultrasonic emission is preferably carried out over a frequency range between 1 MHz and 200 MHz, and more specifically between 5 MHz and 50 MHz.

Said low-frequency elastic waves are advantageously generated over a frequency range between 5 Hz and 2000 Hz.

In accordance with a preferred implementation of the invention, low-frequency elastic waves are generated by mechanical vibration, by radiation pressure, by hyperthermia or by natural vibration of tissues or any other type of energy able to generate low-frequency vibration(s).

Further characteristics and advantages of the invention will emerge from the description below, given by way of non-limiting example, and from embodiments given with reference to the accompanying figures, in which:

FIG. 1 is a schematic view of a device for elastography according to the prior art,

FIG. 2 is a schematic view of implementation of a MED, of which the threadlike tube forms a catheter or an endoscope,

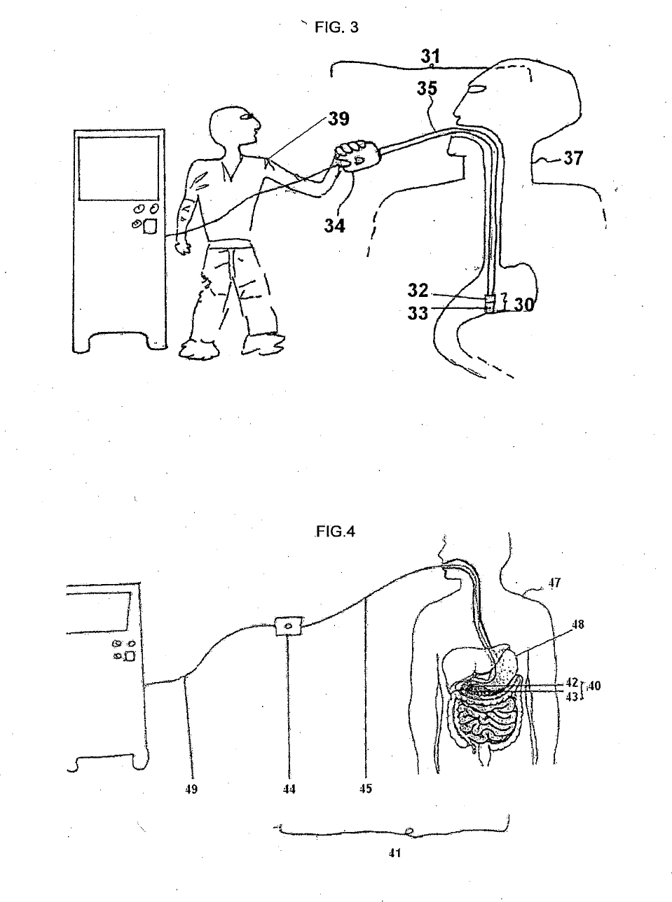

FIG. 3 shows a MED according to the invention, implemented within the human gastrointestinal tract for measurement of PVs relative to the pancreas with the aid of a catheter or an endoscope,

FIG. 4 shows an implementation of a MED according to the invention in the human gastrointestinal tract for the measurement of VPs relative to the stomach or the liver with the aid of an endoscope,

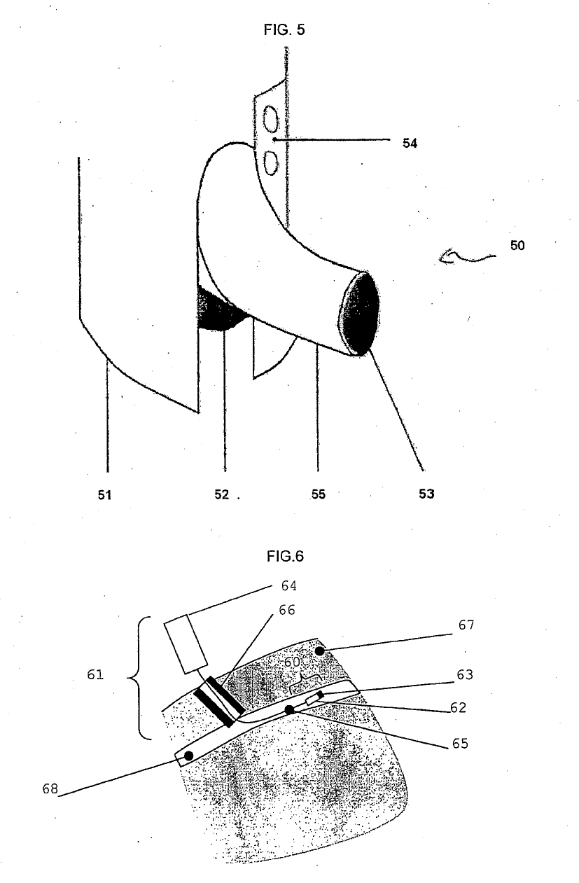

FIG. 5 shows a detail of a probe of a MED according to the invention for an endoscopic application,

FIG. 6 shows a MED according to the invention, of which the threadlike tube forms a catheter,

FIG. 7 is a schematic view of a MED according to the invention implemented in the groin of the leg for measurement of VPs relative to the heart and/or the blood with the aid of a catheter,

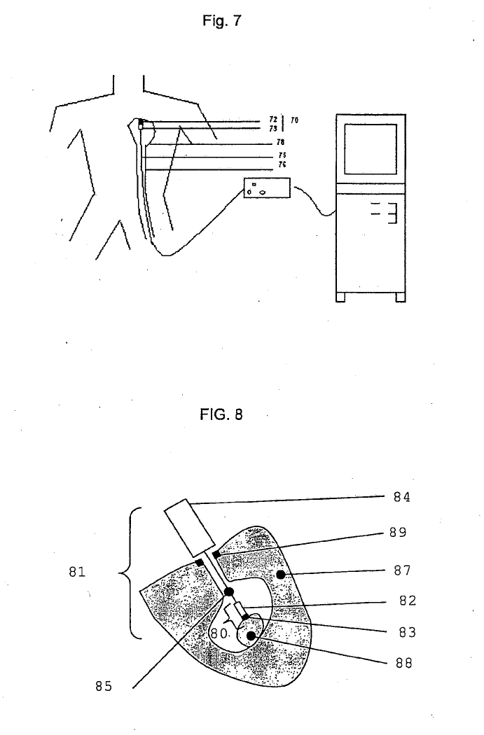

FIG. 8 is a schematic view of the implementation of a MED, of which the threadlike tube forms a laparoscope,

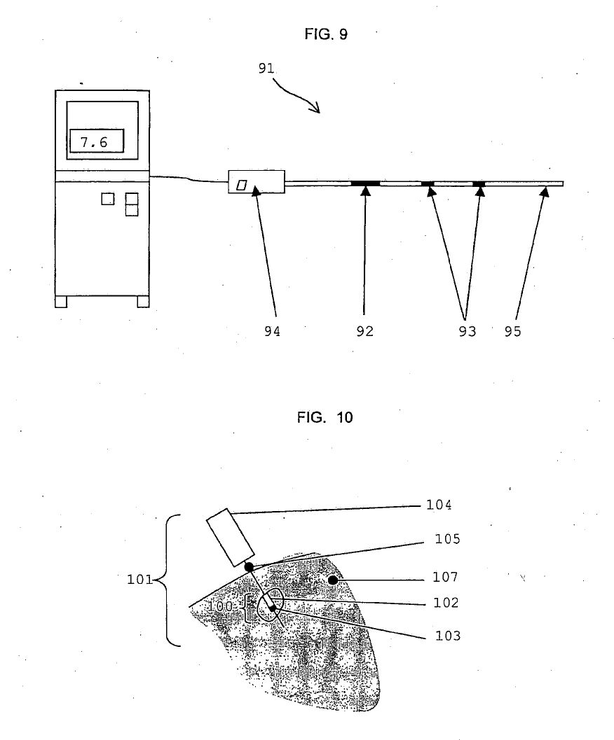

FIG. 9 shows implementation of a device according to the invention for establishing the VPs of tissues belonging to the abdominal cavity,

FIG. 10 is a schematic view of implementation of a MED, of which the threadlike tube forms a needle,

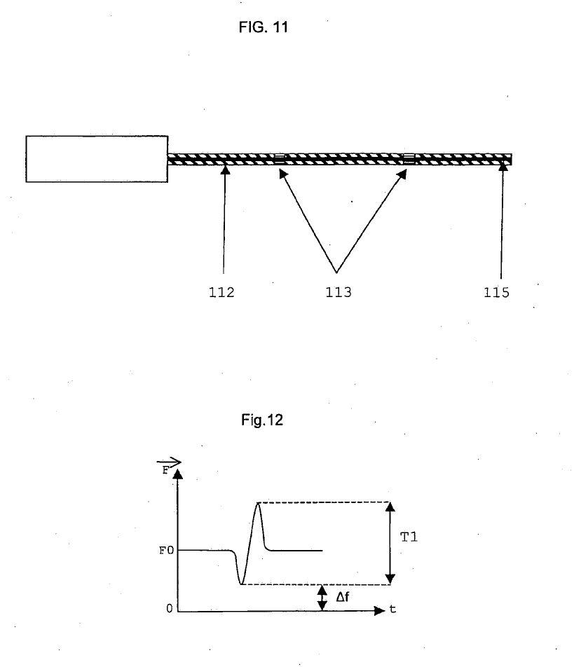

FIG. 11 shows a probe adapted for different types of needle,

FIG. 12 is a graph showing the force exerted on the tissue by the probe.

In the rest of the description vibration elastography is understood to mean a technique for measuring VPs, in which a vibration generator generates, by direct or indirect contact with a tissue, one or more low-frequency elastic waves propagating in this tissue.

The shape of this low-frequency elastic wave over time may be arbitrary, but is more generally of the impulse, transitional or periodic (continuous, monochromatic) type.

This vibration is generally obtained mechanically, but may also be obtained by radiation pressure, by ultrasonic hyperthermia or by vibrations within the body (heartbeats, pulse, etc.). Likewise, the vibration may also be obtained with the aid of a vibration generator arranged outside the body.

Furthermore, different types of mono-element or multi-element ultrasonic transducers may be used in the MED of the invention. For example and in a non-limiting manner, the ultrasonic transducer may be a crown, annular, 2D matrix, linear or convex strip transducer, a mono-element transducer, a tri-element transducer, or a star-type transducer, etc.

By way of example and with reference to FIG. 2, a device 21 for micro-elastography according to the invention is equipped with a probe 20, formed of a vibration generator 22 generating low-frequency elastic waves and at least one ultrasonic transducer 23, which probe is connected to a controller 24, kept outside the body 27, by a flexible threadlike tube 25 free from any angular stresses, more precisely spatially mobile with no mechanical stress owing to its ductility, in particular suppleness and flexibility.

The probe is transported via a natural route 26, such as the gastrointestinal tract, against an organ 28 belonging to the human or animal body, the viscoelastic properties of which it is sought to establish. This implementation advantageously makes it possible to contact organs deep within the human or animal body via natural routes, that is to say in a non-invasive manner, so as to establish precisely the qualitative and/or quantitative data relative to the viscoelastic properties.

Referring to FIG. 3 a device 31 for micro-elastography according to the invention is equipped with a vibration generator 32 generating low-frequency elastic waves and at least one ultrasonic transducer 33 controlled by an external controller 34.

The vibration generator 32 and the ultrasonic transducer 33 are connected to the controller 34 by a flexible threadlike tube 35 free from angular stresses, making it possible to insert the vibration generator 32 and at least one ultrasonic transducer 33 into the human body 37 whilst the controller 34 is kept outside the body.

A user 39 using the MED 31 to acquire data may thus lead the probe 30 of this MED near to or against deep tissues so as to measure their VPs, overcoming the drawbacks lined to the presence of an adipose layer in the vicinity of the epidermis or to the diffraction of waves emitted, as described previously with the prior art.

As shown in FIG. 4, a human being has many natural routes enabling guidance of this type of the probe of a MED according to the invention in an organism, in particular the upper airways—such as the nose or mouth—and the lower routes—such as the rectum—make it possible to insert a probe of a MED into the human body.

For example the probe 40 of a MED 41 may be inserted via the upper airway of a human 47 so as to be transported, thanks to the supple and flexible threadlike tube 45 and via the gastrointestinal tract, into the stomach 48. When the probe 40 is near to or against the tissue to be analysed, a mechanical vibration is generated by the vibration generator of the probe 40 so as to transmit one or more low-frequency elastic waves in this tissue to be analysed.

In order to follow the displacement of this or these low-frequency elastic waves, the ultrasonic transducer of the probe 40 emits and simultaneously acquires high-frequency ultrasonic waves. The ultrasonic signals received are processed so as to measure the displacements produced in said medium by this or these low-frequency elastic waves.

The spatio-temporal development of these displacements makes it possible to obtain quantitative and qualitative data relative to the VPs of the tissue analysed in accordance with elastography techniques.

The vibration generator and the ultrasonic transducer are controlled by a controller 44 arranged outside the body of the patient 47. More precisely, this controller 44 makes it possible to control a power source transmitting the energy required to operate the vibration generator or ultrasonic transducer. This energy is therefore transferred to the controller 44 via a wire connection 49. This energy is transferred from the controller 44 to the vibration generator and to the ultrasonic transducer via the mechanical connection means formed by a threadlike tube 45.

The MED 41 makes it possible to measure the VPs of tissues against which it is contacted directly, and also the VPs of tissues arranged in the vicinity of these contacted tissues.

For example said probe 40 of a MED 41 is contacted with the wall of the stomach 48 of the patient 47. This spatial configuration offers the possibility of establishing the VPs of the tissue of the stomach 48, and also the VPs of the liver since these two organs—the liver and stomach—are close, near to one another and in direct or indirect contact with the probe 40.

To summarise and in general, the probe is inserted into a body, for example via a natural route, until one of its ends is in direct or indirect contact with a tissue, of which the VPs are to be established.

Following this direct or indirect contact, a method of vibration elastography is used. A low-frequency vibration of arbitrary, impulse, transitional or periodic type is generated by using a generator of a low-frequency elastic wave in the biological tissues and by measuring, via at least one ultrasonic transducer, the response of the biological tissue to this or these low-frequency elastic wave(s).

The MEDs 31, 41 described previously may be implemented in the form of catheters or endoscopic devices, the probes 30 and 40 forming the distal end of the endoscopic device, whilst the controller 34 or 44 is arranged at the proximal end.

The MEDs 31 or 41 described previously may also be implemented in the form of accessories intended, inter alia, to be inserted into the operating channel of an endoscope, the probes 30, 40 forming the distal end of the accessory whilst the controller 34 or 44 is arranged at the proximal end.

In particular FIG. 5 shows an implementation of a MED according to the invention in the form of an accessory inserted into the operating channel of an endoscope, the distal end of which accessory comprises a probe 50 adapted for endoscopy and formed of a transducer 53 and a vibration generator (not referenced). In this embodiment the threadlike tube 55 is inserted through the operating channel of a duodenoscope 51 equipped with an erector 52, the main task of which is to orientate and position the probe 50 in front of the tissues to be measured. The endoscope 51 ideally also comprises a display system 54, for example optical or ultrasonographic, so as to make it possible to guide the distal end of the MED to the tissues of which the VPs are to be measured. The display system attached to the MED thus facilitates transportation and also its positioning and offers the practitioner the possibility of ensuring that the tissue displayed corresponds to the desired tissue.

A display system of this type makes it possible to confirm the perpendicularity of the probe to the tissue to be measured with a view to ensuring optimal propagation of elastic waves in the tissues. In fact, propagation of the low-frequency wave in a direction different to that of the ultrasounds is not conducive to producing a reliable measurement of its speed and therefore of VPs of human or animal biological tissue.

In accordance with a different implementation shown in FIG. 6 a micro-elastography device 61 according to the invention is equipped with a probe 60 formed of a generator of (a) low-frequency elastic wave(s) 62 and at least one ultrasonic transducer 63, which probe is connected to a controller 64, kept outside the body 67, by a flexible threadlike tube 65 free from any angular stress. According to this implementation, said probe 60 is first inserted through a route formed by an instrument 66, for example a trocar or a cannula, thus then allowing insertion of said probe 60 within a natural route 68.

By way of example and with reference to FIG. 7, an embodiment of the invention implementing a threadlike tube formed by a catheter 75 is described, said threadlike tube acting as a mechanical connection and guidance means having a suppleness required to follow blood vessels without damaging them. Furthermore, its small diameter, typically less than 3 mm, is adapted so as to be inserted into blood vessels. A MED of this type may be inserted into an artery, a vein, a capillary or any other type of vessel, for example at the top of a thigh—the groin 76—or an arm, or at a jugular vein so as to establish the VPs of the walls of said blood network, of the blood or of an organ such as the heart 78.

In addition, as shown in FIG. 8, a device according to the invention 81 of micro-elastography is equipped with a probe 80, formed of a vibration generator 82 generating low-frequency elastic waves and at least one ultrasonic transducer 83, which probe is connected to a controller 84, kept outside the body 87, by a rigid threadlike tube 85. In accordance with this implementation the probe 80 is transported against or near to the organ 88 via an artificial route 89 formed by an instrument, such as a surgical retractor, a cannula, a trocar or any other type of hollow cylindrical shaft allowing the probe and the threadlike tube to be passed through.

According to a variant offered by the invention the vibration generator may be the threadlike tube 85.

Referring to FIG. 9, the threadlike tube 95 is formed by an endoscope of the flexible or rigid type, such as a laparoscope, and thus makes it possible to see the organs and the tissues of the abdomen.

Furthermore, the ultrasonic transducer(s) 93 and the vibration generator 92 may be positioned at the distal end of the MED or any other point of the threadlike tube, as shown in the figure.

FIG. 10 shows a different implementation, in which a device according to the invention 101 for micro-elastography is equipped with a probe 100, formed of a vibration generator 102 generating low-frequency elastic waves and at least one ultrasonic transducer 103, which probe is connected to a controller 104, kept outside the body 107, by a rigid or supple threadlike tube of the needle type 105. Said needle, transported via percutaneous route, may be a needle of the biopsy type, thus enabling the practitioner to establish the value of the VPs of tissues close to the needle during its insertion so as to guide the practitioner, during transportation of the probe, to the puncture site and enable the practitioner to ensure that the tissue sample, a biopsy of which it is sought to take, corresponds to a tissue of which the VPs have been modified.

According to a variant of the invention not shown, the threadlike tube formed by the needle is the vibration generator, this vibration being transferred by the needle.

According to an additional variant of the invention shown in FIG. 11, the threadlike tube is an accessory such as a hollow shaft 112 comprising at least one ultrasonic transducer 113, which hollow shaft covers a needle 115.

Furthermore, in a general and non-limiting manner, accessories may also be joined to the probe of a MED, as described in the different implementations, for example tweezers, an inflatable balloon, a cutting blade, an optic fibre, a video camera or an ultrasonographic system, etc.

Moreover, in accordance with a possibility offered by the invention a MED according to the invention may be used to establish the effects of a treatment by generating high-intensity focused ultrasounds “HIFU”. This implementation therefore enables the practitioner to know, at any moment, whether the pathological tissue has been destroyed and to decide whether treatment should be stopped.

As indicated previously, an external power source to the MED transfers the energy required to generate a low-frequency elastic wave or waves and ultrasonic waves via a wire connection and then via mechanical connection means.

According to different variants of the invention, the low-frequency wave or low-frequency waves is/are transferred via a system using pneumatic energy, hydraulic energy or electric energy, activating a micro-mechanism arranged in the probe.

The device is advantageously equipped with a device for controlling the position of the end of the MED.

Within the scope of measuring VPs of tissues, a low-frequency elastic wave or low frequency elastic waves between 5 Hz and 2000 Hz and, more generally, between 10 Hz and 1000 Hz is/are produced. So as to follow the displacement of the low-frequency elastic wave or of low-frequency elastic waves and therefore deduce the speed of displacement thereof, a series ultrasonic waves is thus emitted simultaneously with the emission of the low-frequency elastic wave or waves. The ultrasonic shootings are carried out at a time interval varying between 1 us and 100 ms and, more generally, between 100 us and 1 ms. The ultrasonic emissions are associated with receipts of ultrasonic signals constituted by the overlap of the echoes reflected by the diffusers present in the studied medium.

These ultrasonic shootings are generally carried out over a minimum duration corresponding to the period of the low-frequency wave (inverse of frequency), that is to say 0.5 ms for a frequency of 2000 Hz, and may be carried out continuously for the entire duration of the examination. The minimum duration of 1 ms corresponds to the minimum time required to observe the propagation of one or more low-frequency elastic waves so as to establish the viscoelastic properties of the tissue.

The number and speed of the ultrasonic shootings therefore depend on the frequency of low-frequency elastic waves and on the depth measured.

So as to produce a low-frequency elastic wave, it is desirable for the probe to remain in contact with the tissues. By way of comparison it should be noted that an elastographic measurement according to the prior art requires a force of approximately 4 N to 8 N between the generator and the skin so as to ensure propagation of the wave as far as the tissues to be measured. So as to maintain this contact, besides intervention of the user of the MED, a prestressing may be implemented so as to generate a static force F0, as shown in FIG. 12 where the axis of the ordinates represents the force exerted whilst the axis of the abscissa represents time.

As a result, an impulse T1 may be given in such a way that a variation of force is positive (Δf>0) and produces the low-frequency wave whilst still keeping the ultrasonic transducer in contact with the tissues.

According to one possibility offered by the invention, the static force F0 may be maintained and the low-frequency stress may be generated with the aid of one of the following elements: a spring, an elastomer, a pneumatic apparatus, a hydraulic or muscular apparatus or any type of element having the characteristic of maintaining the static force for the entire duration of the acquisition period.

According to a variant not shown, the low-frequency wave is produced differently by a second device inserted within the threadlike tube. This threadlike tube may be the operating channel of an endoscope.

The low-frequency elastic wave or the low-frequency elastic waves may also be produced by an external ultrasonic transducer in radiation pressure mode or by a vibrator arranged outside the body.

According to an additional variant not shown, the elastic waves taken into account are produced by the displacements of organs arranged in the body, such as heartbeats.

A protection, which is transparent to the ultrasounds and of which the characteristics of elasticity are close to those of tissues of which the VPs are to be established, may be arranged on the probe so as to protect said probe against corrosive effects, and/or to observe the required conditions of biocompatibility and sterility, and to reduce the risks of contamination.

Non-limiting examples of the threadlike tube include a catheter, a tube, a pipe, a conduit, a channel, a sheath, an endoscope, a needle and also any other supple, flexible or rigid connection means longer than 20 mm, typically between 20 mm and 3 metres long, making it possible to transport and position the probe in direct contact with or near to the tissue of which the VPs are to be established.

Whilst the probe is intended to be inserted into a body, the external controller and the energy source to which it is connected via a wire connection are kept outside the respective body so as to enable a practitioner to control the transducer and the vibration generator of the probe.

The invention makes it possible to overcome fatty layers in the vicinity of the epidermis in such a way that the measurements of VPs and interpretation thereof are simplified and improved.

In addition, it is known to measure viscoelastic properties over a depth between 25 and 65 mm, which makes it possible to take into account the presence of an adipose layer.

Within the scope of the invention the MED is in direct contact with tissues that are sometimes less than 25 mm thick, and are typically between 2 and 12 mm thick.

In order to make it possible to measure VPs in these tissues close to the probe, the present invention generates low-frequency elastic waves of which the frequency is between 10 Hz and 1000 Hz, that is to say at a frequency that is higher than the frequency conventionally implemented to measure viscoelastic properties using a probe outside the human body—approximately 50 Hz.

Moreover, the small thickness of tissues to be analysed according to the invention also raises a problem of resolution. The present invention therefore proposes increasing the frequency of the ultrasonic waves used so as to follow the low-frequency elastic waves. This frequency is between 1 MHz and 200 MHz and, more specifically, between 5 MHz and 50 MHz so as to obtain increased resolution relative to the prior art.

The MED according to the invention is advantageously controlled by at least one computer, a micro-computer, a central unit or any type of control system, thus making it possible to adapt the frequency of the low-frequency elastic wave or waves produced within the depths of tissues that it is wished to display. Thanks to this specific detail, the present invention proposes a MED that makes it possible to obtain a low-frequency vibration, or impulse, that is perfectly controlled with regard to time and amplitude and is adapted to the thickness to be measured.

The present invention is described above by way of example. It is understood that the person skilled in the art is able to produce different variants of the invention without departing from the scope of the patent. Different methods thus make it possible to obtain qualitative measurements of VPs of tissues with the aid of statistical treatment of a plurality of measurements. For example values that deviate, to a predetermined extent, from the mean value of these measurements can be rejected.

1. A device for vibration elastography for the quantitative and/or qualitative measurement of viscoelastic properties of a human or animal tissue, the device comprising:
a probe comprising at least one ultrasonic transducer and a low-frequency vibration generator, the at least one ultrasonic transducer configured to generate ultrasonic waves making it possible to analyze the propagation of low-frequency elastic waves propagating in the organ and generated by the low-frequency vibration generator, the probe being intended to be positioned near to or against the organ;
a controller, connected to the probe, and configured to actuate the probe, the controller being intended to be kept outside the human or animal body;
a wire connection configured to transfer energy to the controller; and
a threadlike tube constructed and arranged to mechanically connect the probe to the controller.

2. The device according to claim 1, wherein the threadlike tube is longer than 20 mm, and is preferably between 20 mm and 3 metres long.

3. The device according to claim 1, wherein the threadlike tube is flexible and free from angular stresses.

4. The device according to claim 1, wherein the threadlike tube is rigid.

5. The device according to claim 1, wherein the threadlike tube is the vibration generator.

6. The device according to claim 1, wherein the ultrasonic transducer has an active diameter of less than 3 mm.

7. The device according to claim 1, wherein the threadlike tube forms a catheter, of which the distal end contains the probe and the proximal end comprises the controller.

8. The device according to claim 1, wherein the threadlike tube forms a needle, of which the distal end contains the probe and the proximal end comprises the controller.

9. The device according to claim 1, wherein the threadlike tube forms an endoscope, of which the distal end contains the probe and the proximal end comprises the controller.

10. The device according to claim 1, wherein the threadlike tube forms a hollow shaft into which a needle is inserted.

11. The device according to claim 1, wherein the controller is constructed and arranged to control the transfer of energy to the vibration generator and/or to the at least one ultrasonic transducer.

12. A method for vibration elastography for the quantitative and/or qualitative measurement of viscoelastic properties of a human or animal tissue, implementing a device according to claim 1, the method comprising:
positioning the probe near to or against the tissue to be measured;
keeping in contact with the tissue at least one ultrasonic transducer while the step of ultrasonic emission and acquisition and of generation of one or more low-frequency elastic wave(s) is carried out;
generating one or the low-frequency elastic wave(s);
generating, at the same time as the preceding step, ultrasonic emissions and acquiring, at high speed, high-frequency ultrasonic signals during propagation of the low-frequency elastic wave or waves;
calculating the spatio-temporal variations of displacements and/or deformations and/or speeds of displacement and/or speeds of deformation produced in the organ or, more generally, of any movement parameter; and
calculating the viscoelastic properties of the tissue.

13. The method according to claim 12, wherein the probe and a portion of the threadlike tube are inserted via a natural route into a human or animal body.

14. The method according to claim 12, wherein the probe and a portion of the threadlike tube are inserted via percutaneous route into a human or animal body.

15. The method according to claim 12, wherein the probe and a portion of the threadlike tube are inserted via artificial route or via material route into a human or animal body.

16. The method according to claim 12, wherein the threadlike tube is inserted into the operating channel of an endoscope and is oriented using an erector equipping the endoscope so as to position the distal end of the device in front of the tissues to be measured.

17. The method according to claim 12, wherein the probe and a portion of the threadlike tube are inserted into a liquid belonging to the human or animal body.

18. The method according to claim 12, wherein the high-frequency ultrasonic emission is carried out over a frequency range between 1 MHz and 200 MHz.

19. The method according to claim 12, wherein low-frequency elastic waves are generated over a frequency range between 5 Hz and 2000 Hz.

20. The method according to claim 12, wherein low-frequency elastic waves are generated by mechanical vibration, by radiation pressure, by hyperthermia or by natural vibration of tissues.

21. The device according to claim 2, wherein the threadlike tube is between 20 mm and 3 meter long.

22. The method according to claim 18, wherein the high-frequency ultrasonic emission is carried out over a frequency range between 5 MHz and 50 MHz.
